(19) United States
(12) Patent Application Publication
(10) Pub. No.: US 2004/0131575 A1
(43) Pub. Date: Jul. 8, 2004

(54) Stain removing composition

(76) Inventor: Kari Serruys, Chatsworth, CA (US)

Correspondence Address: Marvin H. Kleinberg, Suite 1080, 2049 Century Park East, Los Angeles, CA, 90067, US

(21) Appl. No.: 10/741,025

(22) Filed: Dec. 19, 2003

Related U.S. Application Data
(60) Provisional application No. 60/435,411, filed on Dec. 20, 2002.

(51) Int. Cl. A61K 7/075; A61K 7/08

(52) U.S. Cl. 424/70.22

(57) ABSTRACT

A composition suitable for removing the stains caused by self tanning products which include a coloring agent and DHA is comprised primarily of a mild, unsaturated fatty acid surfactant. In a preferred embodiment, a mild abrasive powder and a thickening agent are added to expedite the stain removal resulting in a gel or paste which can be dispensed from containers. Other embodiments can be more liquid for spray application or utilize pads wetted with the composition.

### CROSS REFERENCE TO RELATED APPLICATIONS

This is a continuation-in-part of U.S. Patent Application Serial No. 60/435,411, filed Dec. 20, 2002, the priority of which is claimed herein.

### STATEMENT REGARDING FEDERALLY SPONSORED RESEARCH OR DEVELOPMENT

Not Applicable

### REFERENCE TO A SEQUENCE LISTING, A TABLE, OR A COMPUTER PROGRAM LISTING COMPACT DISK APPENDIX

Not Applicable

### BACKGROUND OF THE INVENTION

1. Field of the Invention

The present invention relates to skin care products and, more particularly, a composition for gently and effectively removing cosmetic stains from human skin.

2. Description of the Related Art

More consumers are becoming aware of the skin damaging effects of ultra-violet radiation from repeated sun exposure, causing “instant self-tanning” or “instant sunless tanning” products to gain popularity in recent years. Most such products contain the active ingredient dihydroxyacetone (DHA) which reacts with protein in skin's stratum corneum to produce a brown color somewhat similar to the color of suntan. The first patent on the use of DHA as a skin coloring agent (U.S. Pat. No. 2,949,403) was issued to J. Andreadis in 1960.

One major problem associated with DHA-based self tanning products is the difficulty of even application. An aqueous solution of DHA is colorless and its reaction with skin to produce brown color is quite slow. Since it takes several hours for the color to become visible and about 24 hours to complete color development, it is difficult for the user to know if the solution has been correctly applied to the desired areas of the body or if the application is even.

To minimize this problem, many DHA-based self-tanning products now contain coloring agents to assist the users in identifying areas of uneven or unwanted application. The coloring agents may consist of any combination of food colors, organic dyes or inorganic pigments. However, the added coloring agents can have the unintended effects of staining areas which should not “tan,” such as the palms or fingernails. More important, the coloring agents can indicate those areas that will be affected by the DHA over time. In this way, the user can quickly spot areas from which the DHA should be removed before any reaction can occur.

When combined with DHA, the color stain can be very difficult to remove with ordinary soap and water, particularly if the color is deep or if it is allowed to stay on the skin for a period of time, (preferably less than the time required for the DHA to react with the skin). Even after repeated washing with soap and water, complete removal may be difficult.

Use of a stronger detergent or considerable scrubbing may be needed. Other methods of stain removal exist, however, use of a strong detergent, i.e. sodium lauryl sulfate, or repeated scrubbing with a brush, can be injurious to skin or cause severe drying of the skin. As a result, there is a need for a “stain remover” that is both safe and easy to use, as well as having the ability to remove easily the skin staining colorants and the DHA found in instant sunless tanning products.

### SUMMARY OF THE INVENTION

According to a preferred embodiment of the present invention, a stain remover consisting of a mild, unsaturated fatty acid surfactant, such as ammonium oleate has been found to have the unique combination of properties required to remove stains created by instant sunless tanning products safely and effectively. Unsaturated fatty acid surfactants are also suitable for use with mild abrasive scrubbing agents such as low-density polyethylene powder or finely ground pumice.

In the preferred embodiment of the invention, an unsaturated fatty acid surfactant is combined with an abrasive and a compatible hydrophilic polymeric thickener to form a gel suitable for packaging in a tube, jar, bottle, vials, can, packette or other suitable product container compatible with these formulations.

The package container may include a spray head, squirt nozzle, dropper or similar device intended to make dispensing and application easier for the user. The stain remover may also be incorporated into an absorbent pad and packaged either as in individually wrapped disposable packettes for one-time use or in a larger, resealable container containing multiple absorbent pads. Pads also have the advantage of providing mild abrasive action, assisting the activity of the unsaturated fatty acid surfactant.

The resulting product is an easy to use and effective stain remover, particularly suitable for removing stains on skin resulting from coloring agents that are mixed with DHA-based self-tanning products.

Unsaturated fatty acids surfactants or soaps, such as oleic acid soaps, are relatively mild on skin but they are not regarded as effective a cleansing agent as are saturated fatty acid soaps or synthetic detergents, which are used commonly in household soap bars.

Common bar soaps consist of a mixture of sodium or potassium salts of saturated lauric, myristic, palmitic, stearic or coconut fatty acids. They may also contain synthetic detergents such as sodium cocoyl isethionate.

The addition of a mild abrasive powder to unsaturated fatty acid acids soaps together with a suitable polymer thickener in the preferred system results in a gel or paste that is convenient to use. Such a combination is very effective in removing stains caused by the coloring agents which are used in combination with DHA-based self-tanning products. It was surprising to discover that these relatively poor surfactants could remove colorants that were specifically chosen for their long-lasting ability to stain the skin.

In addition, the present invention is also quite effective in removing the DHA-based tanning agents. If done soon enough after application, the DHA component is removed before it has had an opportunity to react with the skin and achieve the tanning effect.

In an alternative embodiments, the formulation is prepared without the mildly abrasive powder or the thickener. These embodiments could be used with a textured cloth or fibrous material, i.e. absorbant paper towelettes. In use, the applicator is sufficiently abrasive in combination with the mild surfactant to remove the stains. It has been found that, in some instances, where a formula based on this invention is sprayed on the surface, rubbing with the fingers provides sufficient abrasion to effect a removal of the stain, contrary to expectations.

### DESCRIPTION OF THE PREFERRED EMBODIMENTS

As required, a detailed illustrative embodiment of the present invention is disclosed herein. However, manufacturing techniques, choice of ingredients, instructions for use etc. for the present invention may be embodied in a wide variety of forms and modes, some of which may be quite different from those in the disclosed embodiment. Consequently, specific choice of preferred ingredients and other functional details disclosed herein are merely representative and were chosen to afford the best embodiment for purposes of disclosure and to provide a basis for the claims and to define the scope of the present invention.

According to a preferred embodiment of the present invention, a combination of deionized water (85.80% by weight) is mixed with disodium EDTA (0.10% by weight) and ammonium hydroxide (28-30%) (1.50% by weight) to produce a first, Phase A of the formulation. A second, PHASE B is comprised of oleic acid (7.00% by weight), fragrance oil (0.30% by weight), propylparaben (0.10% by weight) methylparaben (0.20% by weight), polyethylene powder (4.00% by weight) and Structure 2001 (1.00% by weight). Phase A and Phase B combine for a total of 100.00%

The disodium EDTA and ammonium hydroxide are added to a stainless steel tank containing deionized water, resulting in PHASE A. Care must be taken so that all solids are completely dissolved in the water.

In another stainless kettle the PHASE B ingredients are added, starting with oleic acid, fragrance oil, propylparaben and methylparaben which are mixed and dissolved. Next, combine PHASE A ingredients with the PHASE B ingredients of the other kettle, thoroughly mixing them together while avoiding air entrapment. Next add the polyethylene powder and mix to uniformity. Finally, add Structure 2001 and mix to uniformity, again avoiding air entrapment. The resulting composition has a gel like consistency that can then be packaged either in a tube, a squeeze bottle, or other herein described container for easy dispensing.

In alternative embodiments, either the sodium or potassium salts of oleic acid, or the ammonium, sodium or potassium salts of erucic acid or ricinoleic acid may be used as the surfactant or soap base at concentrations ranging from 1 to 40%.

Although the polyethylene powder is preferred since it is a gentle and mild abrasive for use on the skin, other more abrasive powders could be substituted, i.e. walnut shell powder, almond meal powder, apricot seed powder, luffa or other mildly abrasive powders, could be substituted.

These alternative powders can be used at concentrations ranging from 0.1% to 50%. Alternatively, acrylic polymers, such as Carbopol ETD 2020 (Acrylates/C10-30 Alkyl Acrylate Crosspolymer) from B. F. Goodrich Specialty Chemicals (now Noveon) can also be used for this purpose at concentrations ranging from 0.1% to 20%.

Although polymers are the preferred thickener, specifically acrylic polymers, other cosmetic thickeners and rheological additives known to those skilled in the art of formulating such products may employed to achieve the gel or paste consistency required to thicken the fatty acids soaps and suspend the mild abrasive particles, i.e. polyethylene powder, clay, silica, cellulosic, gum, glycol, etc.

In other alternative embodiments, the formulation, without the abrasive powders can be applied to absorbent cloth, paper or fiber pads which, when used as the applicator, are sufficiently abrasive to obviate the need for the powders. The pads or towelettes can be packaged in moisture proof containers and used, much in the same fashion as the ubiquitous towelette generally used for hand and face washing in eating places.

However, in alternative embodiments, the thickeners can be dispensed with, leaving only the mild unsaturated fatty acid surfactants as the active ingredient which need only a mildly abrasive action to remove stains and the DHA. These embodiments can be conveniently packaged in spray-on containers.

It can be seen that a mild unsaturated fatty acid composition even when not combined with a mildly abrasive powder can be effective in removing the stains caused by coloring agents present in DHA based self-tanning compounds. At the same time, the DHA component will be removed as well, thereby preventing the staining and “tanning” of areas of skin for which “tanning” is an undesired result of the self-tanning process.

Thus there has been described a formulation that easily removes self tanning compositions from areas of the body that have been unintentionally stained. While the preferred embodiment is formulated with a mildly abrasive material, alternative embodiments can rely on the applicator or even fingers to provide the necessary mechanical action that the abrasive component provides in the preferred embodiment of the invention.

Modifications and variations will occur to those skilled in the art. Accordingly, the invention should be limited only by the breadth of the claims appended below.

What is claimed as new is:

1. The new use of an unsaturated fatty acid surfactant comprising the steps of:
a. applying an unsaturated fatty acid surfactant to a skin surface to which there has been applied a staining colorant; and
b. applying mildly abrasive activity to the surfactant to facilitate the removal of said staining colorant from the skin surface,
whereby a mild surfactant is employed to remove a staining colorant from a human skin.

2. The new use of claim 1 wherein the skin surface to be treated has had DHA applied, as well.

3. The new use of claim 1 wherein the saturated fatty acid surfactant has added thereto a mildly abrasive composition to assist in effecting said mildly abrasive activity.

4. A composition for removing staining colorants from human skin comprising an unsaturated fatty acid surfactant.

5. The composition of claim 4 wherein said surfactant is a salt of oleic acid selected from the group including ammonium, sodium and potassium.

6. The composition of claim 4 wherein said surfactant is a salt of erucic acid selected from the group including ammonium, sodium and potassium.

7. The composition of claim 4 wherein said surfactant is a salt of ricinoleic acid selected from the group including ammonium, sodium and potassium.

8. The composition of claim 4, further comprising a hydrophilic polymeric thickener compatible with said unsaturated fatty acid surfactant.

9. The composition of claim 8, further comprising mildly abrasive powder.

10. The composition of claim 9 wherein said powder is polyethylene.

11. The composition of claim 8 wherein said powder is selected from the group comprising walnut shell powder, almond meal powder, apricot seed powder, and finely ground pumice.

12. A device for removing unwanted staining colorant and DHA from human skin surfaces comprising:
a. a stain removing composition including an unsaturated fatty acid surfactant; and
b. delivery means including an absorbant pad impregnated with said surfactant;
whereby lightly rubbing the skin with said pad applies said surfactant to the skin surface thereby removing the colorant stain and DHA from the skin surface being rubbed.

13. The device of claim 12 wherein said stain removing composition further includes a mildly abrasive powder.

14. The device of claim 13 wherein said mildly abrasive powder is polyethylene.

15. The device of claim 13 wherein said mildly abrasive powder is selected from the group including walnut shell powder, almond meal powder, apricot seed powder, and finely ground pumice.

16. The composition of claim 13 further comprising a hydrophilic polymer thickener compatible with said stain removing composition.

17. The composition of claim 12 wherein said absorbant pad impregnated with said surfactant is paper.

18. The composition of claim 12 wherein said absorbant pad impregnated with said surfactant is cloth.

19. The composition of claim 12 wherein said absorbant pad impregnated with said surfactant is an unwoven synthetic fiber.

20. A composition for removing staining colorants from human skin comprising in combination:
a. The ammonium salt of oleic acid;
b. Propylparaben;
c. Methylparaben; and
d. Polyethylene powder;
whereby the ingredients are thoroughly mixed into a gel consistency for topical application to the skin.
